(19) United States
(12) Patent Application Publication
(10) Pub. No.: US 2022/0003730 A1
(43) Pub. Date: Jan. 6, 2022

(54) GAS SENSOR DEVICE

(71) Applicant: Panasonic Intellectual Property Management Co., Ltd., Osaka (JP)

(72) Inventors: KIYOKAZU ITOI, Osaka (JP); SHINYA SUZUKI, Kyoto (JP)

(21) Appl. No.: 17/342,977

(22) Filed: Jun. 9, 2021

(30) Foreign Application Priority Data
Jul. 2, 2020 (JP) 2020-114785

(51) Int. Cl. G01N 33/00

(52) U.S. Cl. CPC G01N 33/0011

(57) ABSTRACT

A gas sensor device includes: a substrate having a cavity structure; a gas sensor disposed in a recess portion of the cavity structure of the substrate; and a thin film bonded to the substrate, covering the recess portion, and being impermeable to liquid and permeable to gas.

### BACKGROUND

### 1. Technical Field

The present disclosure relates to a gas sensor device for detecting gas molecules containing specific atoms in gas.

### 2. Description of the Related Art

A gas sensor represents an element that reacts with gas to detect existence of the gas or converts a gas concentration into an electric signal or the like and outputs the electric signal. Gas sensors are generally known to be used for gas leak alarms such as those found in homes, gas leak detection for pipelines in infrastructure facilities and factories, and control of fuel cell vehicles. Hereinafter, a typical hydrogen gas sensor among the gas sensors currently in use will be described.

Hydrogen energy is positioned as a most important energy source because the hydrogen energy is highly energy-efficient, and can significantly reduce CO₂ emissions, and water, which is a raw material of the hydrogen energy, can be obtained anywhere. However, on the other hand, the hydrogen energy is also a high-risk energy source. It is widely known that hydrogen gas is dangerous because hydrogen gas has small minimum ignition energy and a wide combustion concentration range, so that hydrogen gas is easily ignited, further has a very high maximum combustion speed, and has a high blast pressure. For these reasons, high reliability and high durability are important for hydrogen gas sensors. There are sensor elements of various principles that are being currently in practical use, such as a semiconductor type, a contact combustion type, and an optical type, and the development of sensors having a fast response speed, high sensitivity, low power consumption, and high environmental resistance is still underway.

Here, an outline of a gas sensor device in Japanese Patent No. 5507524 (PTL 1) will be described with reference to FIGS. 6A to 6C. FIGS. 6A to 6C are explanatory views of the gas sensor device described in PTL 1. FIG. 6A is a cross-sectional view of a main portion of the gas sensor device, FIG. 6B is a bottom view of the gas sensor device, and FIG. 6C is a perspective view of a component for crimping a water-proof moisture permeable material of the gas sensor device. As shown in FIGS. 6A and 6B, the gas sensor device in PTL 1 includes sensor chip 101. Sensor chip 101 is disposed on stem 102 via heat insulating member 103. Sensor chip 101 is attached to an upper surface of heat insulating member 103. Stem 102 includes a plurality of lead terminals 104 that penetrate stem 102 and project from both a front surface and a back surface of stem 102. Lead terminal 104 is fixed to stem 102 by glass material 102a provided on an outer periphery of lead terminal 104. A plurality of electrode pads (not shown) formed on a main surface of sensor chip 101 and the plurality of lead terminals 104 connected to stem 102 are connected by wires 105, respectively.

Further, sensor chip 101, heat insulating member 103, and the plurality of wires 105 are covered with cylindrical metal cap 106. A lowermost portion of a side portion of metal cap 106 (brimmed portion 106a) is bonded to a periphery of stem 102 by welding. Since sensor chip 101 detects hydrogen gas in an outside air, hole 108 through which the outside air is introduced inside metal cap 106 is formed in metal cap 106. Hole 108 is formed substantially in a center portion of an upper portion of metal cap 106. Further, inside metal cap 106, water-proof moisture permeable material 109 is disposed in contact with the upper portion of metal cap 106. Water-proof moisture permeable material 109 is crimped by cylindrical heat insulating member 110 shown in FIG. 6C.

### SUMMARY

A gas sensor device of the present disclosure includes: a substrate having a cavity structure; a gas sensor disposed in a recess portion of the cavity structure of the substrate; and a thin film bonded to the substrate, covering the recess portion, and being impermeable to liquid and permeable to gas.

### BRIEF DESCRIPTION OF THE DRAWINGS

FIG. 1 is an overall cross-sectional view of a gas sensor device according to a first embodiment of the present disclosure;

FIG. 2 is a schematic perspective view of the gas sensor device according to the first embodiment of the present disclosure;

FIG. 3 is an overall cross-sectional view of a gas sensor device according to a second embodiment of the present disclosure;

FIG. 4 is a plan view of the gas sensor device according to the second embodiment of the present disclosure;

FIG. 5 is an overall cross-sectional view of a gas sensor device according to a third embodiment of the present disclosure;

FIG. 6A is a cross-sectional view of a main portion of a gas sensor device described in PTL 1;

FIG. 6B is a bottom view of the gas sensor device described in PTL 1; and

FIG. 6C is a perspective view of a component for crimping a water-proof moisture permeable material of the gas sensor device described in PTL 1.

### DETAILED DESCRIPTIONS

The gas sensor device in PTL 1 described above has a structure in which hole 108 through which the outside air is introduced toward sensor chip 101 which detects gas is formed in metal cap 106, water-proof moisture permeable material 109 having waterproofness is disposed to prevent water from entering metal cap 106, and water-proof moisture permeable material 109 is crimped by heat insulating member 110. Therefore, when the gas sensor device is operated in a high humidity environment, liquid water does not permeate through water-proof moisture permeable material 109, but moisture in a gaseous state, which is steam, permeates through water-proof moisture permeable material 109, and is present on the surface of sensor chip 101. Accordingly, gas detection ability is lowered, and therefore, it is difficult to implement a highly sensitive gas sensor device.

The present disclosure is to provide a gas sensor device having high detection sensitivity and high-speed reactivity, and capable of guaranteeing operation in a harsh environment.

Hereinafter, embodiments of the disclosure will be described with reference to the drawings. In these figures, a thickness, a length, or the like of each are different from a shape to be implemented from the viewpoint of the drawing. Further, the number of metal pads and wires on a sensor element and the number of terminals and wirings on a bottom portion of a substrate are also set to be easy to show. Further, a material of each member is not limited to a material to be described below.

### First Embodiment

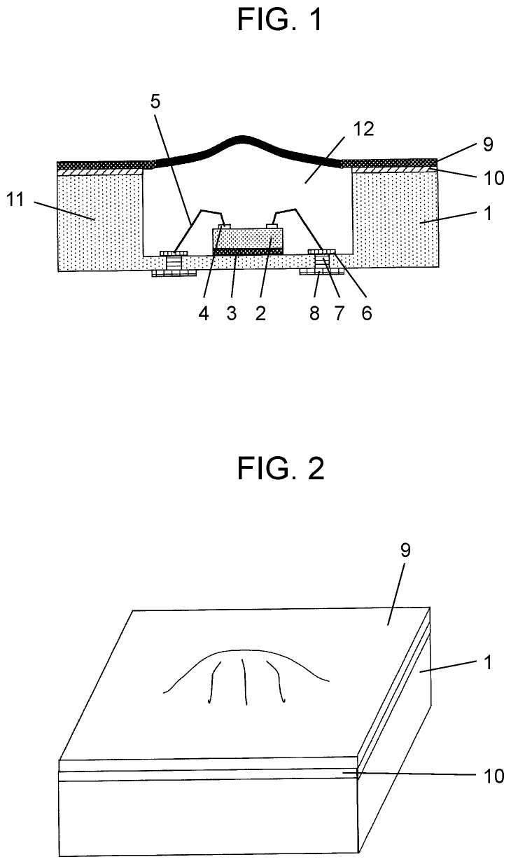

FIG. 1 is an overall cross-sectional view of a gas sensor device according to a first embodiment of the present disclosure, and FIG. 2 is a schematic perspective view of the gas sensor device. A configuration of the gas sensor device of the present disclosure will be described with reference to FIGS. 1 and 2.

As shown in FIGS. 1 and 2, the gas sensor device includes substrate 1. Substrate 1 has a cavity structure formed by outer peripheral wall portion 11 and recess portion 12 surrounded by outer peripheral wall portion 11. Sensor element 2 is fixed to a bottom surface of recess portion 12 of substrate 1 by paste 3. Metal pads 4 are provided on a surface of fixed sensor element 2. Metal pads 4 are electrically connected, via wires 5, to internal terminals 6 provided on the bottom surface of recess portion 12 of substrate 1. Internal terminals 6 are electrically connected, via internal wirings 7 formed inside a bottom portion of recess portion 12 of substrate 1, to external terminals 8 provided on a back surface of substrate 1.

The details of each of the above-described members will be described below.

Substrate 1 is made of a material mainly formed by ceramic. Substrate 1 includes a plurality of layers, and is configured such that the elements mounted on substrate 1 can be electrically connected to the outside of the substrate by forming metal wirings between the layers. An epoxy-based resin or the like may be used for substrate 1, and the material and the structure of substrate 1 are selected according to characteristics and environmental resistance of a product.

Sensor element 2 is an example of a gas sensor that detects gas. A type of sensor element 2 includes a semiconductor element, a microheater element, or the like. A gas detection method includes a method of forming a filament having oxygen defects and detecting a resistance value of a semiconductor element that changes due to adsorption of gas, a method of detecting a change in a resistance value of a microheater element due to a temperature decrease by using a fact that a temperature of the microheater element decreases due to the presence of gas having high thermal conductivity. Due to a difference in the detection principle, characteristics such as power consumption, a measurement limit concentration value, a detection speed, or the like of a device are different. Therefore, it is necessary to select an element according to an application.

Paste 3 functions to fix sensor element 2 to substrate 1, and an epoxy-based resin or a polyimide-based resin is generally used as paste 3. Further, when conduction is required, paste 3 containing Ag fillers can be used, and when insulation is required, paste 3 containing alumina fillers can be used.

Metal pad 4 is required to electrically connect sensor element 2 and internal terminal 6 formed on substrate 1. As a material of metal pad 4, Au, Al, Cu, or the like are mainly used. By connecting metal pad 4 and internal terminal 6 by metal wire 5, metal pad 4 and internal terminal 6 can be electrically connected.

As described above, wire 5 is a wire made of metal necessary for electrically connecting sensor element 2 and substrate 1. As a material of wire 5, Au, Al, Cu, or the like are mainly used. A thickness of wire 5 is about 10 μm to 30 μm. As a wiring method, a method generally called wire bonding is used. In the wire bonding method, for example, an end portion of wire 5 is brought into contact with metal pad 4 formed on sensor element 2, and a first bond side is bonded by applying heat, ultrasonic waves, or a load. Next, an end portion of wire 5 is brought into contact with internal terminal 6 formed on substrate 1 and serving as a second bond side, and the second bond side is bonded by applying heat, ultrasonic waves, or a load. Accordingly, sensor element 2 and substrate 1 can be stably electrically connected.

As described above, internal terminal 6 is a terminal on the second bond side in the wire bonding method. As a material of internal terminal 6, Au, Al, Cu, Ni, Pd, or the like are mainly used. Internal terminal 6 is electrically connected to external terminal 8 via internal wiring 7.

Internal wiring 7 is a metal wiring for electrically connecting internal terminal 6 and external terminal 8 of substrate 1, and is disposed inside substrate 1. As a material of internal wiring 7, metals such as W and Mo are mainly used. In particular, when substrate 1 is made of ceramic or the like, substrate 1 is fired at a high temperature of 1500° C. to 1600° C. in a manufacturing process of substrate 1. Therefore, it is essential that the material of internal wiring 7 is selected such that the material can withstand a high temperature.

External terminal 8 is a metal terminal necessary for conducting electricity of sensor element 2 to substrate 1, and then to an external substrate. As a material of external terminal 8, the same material as internal terminal 6 described above is selected. Solder is placed on a terminal of the external substrate, and external terminal 8 of substrate 1 corresponding to the terminal of the external substrate is aligned with and mounted on the solder, and then heated by a reflow furnace or the like for secondary mounting. Electronic components other than the gas sensor are secondarily mounted on the external substrate by the same method, and a gas sensor module realizes a function of the gas sensor is completed in this way.

Next, a configuration of a front surface side of substrate 1 will be described.

Thin film 9 is bonded to a front surface of substrate 1 via bonding portion 10. A thickness of thin film 9 is, for example, about 10 μm to 50 μm. Thin film 9 is a functional film, and various types of films are used for thin film 9 depending on a purpose of use. Thin film 9 has a function of being permeable to gas and impermeable to water (H₂O in a liquid phase) and moisture (liquid) contained in gas. In order to have such a function, thin film 9 is preferably implemented by a thin film of a water-repellent filter. As a material of the thin film of the water-repellent filter, for example, tetrafluoroethylene, polyester, polyethylene are used. Further, for example, when the gas sensor device is a hydrogen gas sensor device, thin film 9 is preferably implemented by a metal thin film made of Pd or a Pd alloy, a Pd—Cu alloy, TiN, or the like in order to improve gas selectivity while further improving water-proof and moisture-proof functions to enable the gas sensor device to operate even in a high humidity environment, but the material of thin film 9 is not limited to that described above.

As a method for bonding substrate 1 and thin film 9, there are various methods such as ultrasonic bonding using ultrasonic waves, heat, and a load, thermal compression bonding using heat and a pressure, sintering by baking using Ag particles, bonding using a resin which is cured by light or heat. However, in all methods, defects such as tearing of thin film 9 may occur due to vibration and a stress generated by the ultrasonic waves, the heating, the load, or the like during bonding and thermal stress in a subsequent process after the bonding, or the like. In a case of ultrasonic bonding, strain generated by ultrasonic vibration and a load during bonding, and in a case of bonding, a stress generated by adhesive shrinkage during adhesive curing, or the like are typical reasons of the above-described defects. Therefore, a structure is set such that the stress generated in thin film 9 is absorbed by a bulge (slack) provided in a center portion of thin film 9 without stretching thin film 9 when thin film 9 is bonded to substrate 1.

A typical shape of thin film 9 is shown in FIGS. 1 and 2, but a shape of thin film 9 is not limited to the shape shown in FIGS. 1 and 2, and an effect of absorbing the stress generated in thin film 9 is also obtained by a shape in which a bulging portion is not only in the center portion but also in a plurality of portions including or not including the center portion, or a shape (recess, projection, and recess-projection shapes) having bulges and dents such as a swell. Thin film 9 has one or more recess, projection, or recess-projection shapes.

### Second Embodiment

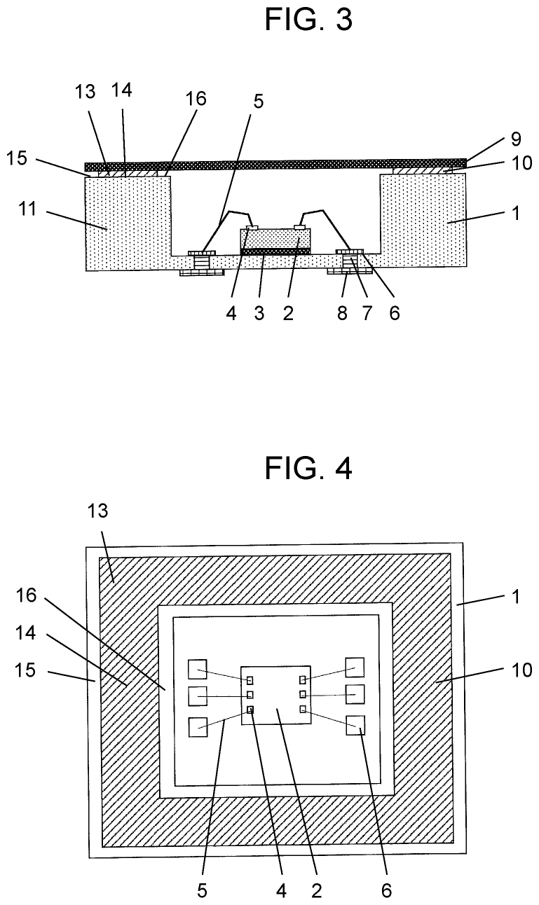

Hereinafter, a second embodiment of the present disclosure will be described with reference to the drawings. FIG. 3 is an overall cross-sectional view of a gas sensor device according to the second embodiment of the present disclosure, and FIG. 4 is a plan view of the gas sensor device according to the second embodiment of the present disclosure. The same configuration as that of the first embodiment may be given the same name and the same reference numeral as that of the first embodiment, and the description thereof may be omitted.

A basic structure of the gas sensor device of the second embodiment is similar to that of the gas sensor device of the first embodiment. Sensor element 2 is fixed by paste 3 to the bottom portion of the recess portion of substrate 1 having a cavity structure. Metal pads 4 are provided on the surface of fixed sensor element 2. Metal pads 4 are electrically connected, via wires 5, to internal terminals 6 provided on the bottom surface of the recess portion of substrate 1. Internal terminals 6 are electrically connected, via internal wirings 7 formed inside the bottom portion of the recess portion of substrate 1, to external terminals 8 provided on the back surface of substrate 1.

The structure in which thin film 9 is bonded to the front surface of substrate 1 is the same as that of the gas sensor device of the first embodiment. However, as shown in FIGS. 3 and 4, bonding portion 10 has a structure in which substrate 1 and thin film 9 are bonded only in center portion 14 (a portion excluding an end portion) of a frame area (upper surface 13 of outer peripheral wall portion 11 in FIG. 3) which is a surface layer of substrate 1, and substrate 1 and thin film 9 are not bonded at outer peripheral edge portion 15 (outer peripheral portion) and inner peripheral edge portion 16 (inner peripheral portion) of the frame area.

The stress generated during bonding of substrate 1 and thin film 9 and in a subsequent process tends to be particularly concentrated on outer peripheral edge portion 15 and inner peripheral edge portion 16 of the frame area, and bonding portion 10 is formed in a shape that avoids outer peripheral edge portion 15 and inner peripheral edge portion 16 of the frame area in order to avoid the stress concentration. Further, in order to further prevent defects such as tearing of thin film 9, it is also more effective to combine a method of bonding substrate 1 and thin film 9 in a state where the center portion of thin film 9 has a bulge as in the gas sensor device of the first embodiment.

### Third Embodiment

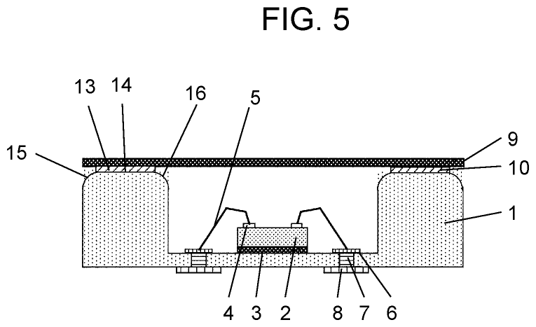

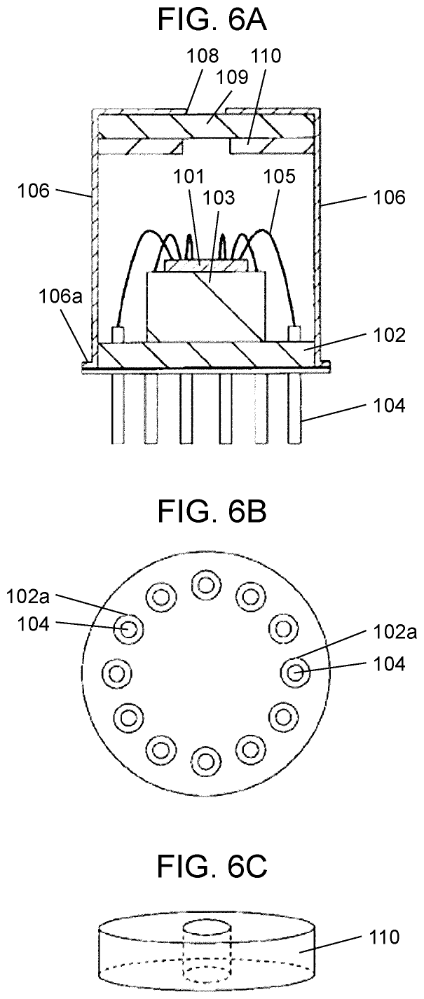

Hereinafter, a third embodiment of the present disclosure will be described with reference to the drawings. FIG. 5 is an overall cross-sectional view of a gas sensor device according to the third embodiment of the present disclosure. The same configuration as that of the first embodiment may be given the same name and the same reference numeral as that of the first embodiment, and the description thereof may be omitted.

A basic structure of the gas sensor device of the third embodiment is similar to that of the gas sensor device of the first embodiment. Sensor element 2 is fixed by paste 3 to the bottom portion of the recess portion of substrate 1 having a cavity structure. Metal pads 4 are provided on the surface of fixed sensor element 2. Metal pads 4 are electrically connected, via wires 5, to internal terminals 6 provided on the bottom surface of the recess portion of substrate 1. Internal terminals 6 are electrically connected, via internal wirings 7 formed inside the bottom portion of the recess portion of substrate 1, to external terminals 8 provided on the back surface of substrate 1.

As shown in FIG. 5, an R shape is provided on an outer peripheral edge portion and an inner peripheral edge portion of a frame area which is a surface layer of substrate 1. Bonding portion 10 is provided in a flat portion other than the R shape in the frame area, and thin film 9 is bonded via bonding portion 10.

As described in the second embodiment, the stress generated during bonding of substrate 1 and thin film 9 and in a subsequent process tends to be particularly concentrated on outer peripheral edge portion 15 and inner peripheral edge portion 16 of the frame area. A surface layer edge portion (outer peripheral edge portion 15 and inner peripheral edge portion 16) of substrate 1 is formed in an R shape in order to prevent peeling and tearing of thin film 9 caused by the stress concentration. That is, the surface layer edge portion of substrate 1 has an R-shaped cross-sectional shape in a thickness direction of substrate 1. Only outer peripheral edge portion 15 or inner peripheral edge portion 16 may be formed in an R shape. Further, in order to further prevent defects such as tearing of thin film 9, it is also more effective to combine a method of bonding substrate 1 and thin film 9 in a state where the center portion of thin film 9 has a bulge as in the gas sensor device of the first embodiment.

The R shape may be provided only on either inner peripheral edge portion 16 or outer peripheral edge portion 15. Further, the R shape has a width of one-third or less of a width of the frame area of substrate 1, and the width may be, for example, narrow or about 0.1 mm, but is not limited to these widths.

According to the gas sensor device of the present disclosure, a gas sensor device having high detection sensitivity and high-speed reactivity and capable of guaranteeing operation in a harsh environment can be provided.

The gas sensor device of the present disclosure is useful in applications that require high characteristics in a harsh environment, such as gas control by sensing inside a fuel cell and detection of leakage in a ground of a buried conduit, which are difficult until now.

What is claimed is:

1. A gas sensor device, comprising:
a substrate having a cavity structure;
a gas sensor disposed in a recess portion of the cavity structure of the substrate; and
a thin film bonded to the substrate, covering the recess portion, and being impermeable to liquid and permeable to gas.

2. The gas sensor device according to claim 1, wherein
a part of the thin film covering the recess portion is loosened.

3. The gas sensor device according to claim 2, wherein
the part of the thin film covering the recess portion has at least one recess shape, at least one projection shape, or at least one recess-projection shape.

4. The gas sensor device according to claim 3, wherein
the part of the thin film covering the recess portion has one recess shape, one projection shape, or one recess-projection shape.

5. The gas sensor device according to claim 1, wherein
the thin film is bonded only to a center portion on an upper surface of an outer peripheral wall portion that defines the recess portion of the substrate.

6. The gas sensor device according to claim 1, wherein
at least one of an inner peripheral portion and an outer peripheral portion of an upper surface of an outer peripheral wall portion that defines the recess portion of the substrate has an R-shaped cross-sectional shape in a thickness direction of the substrate, and
the thin film is bonded only to a flat portion other than an R-shaped portion on the upper surface of the substrate.

7. The gas sensor device according to claim 1, wherein
the thin film is a metal thin film.

8. The gas sensor device according to claim 7, wherein
a material of the metal thin film is any one of Pd, a Pd alloy, a Pd—Cu alloy, and TiN.

9. A gas sensor device, comprising:
a substrate having a cavity structure;
a gas sensor disposed in a recess portion of the cavity structure of the substrate; and
a thin film bonded to the substrate so as to cover the recess portion and impermeable to liquid but permeable to gas, wherein
a part of the thin film covering the recess portion is loosened,
the part of the thin film covering the recess portion has one recess shape, one projection shape, or one recess-projection shape,
the thin film is bonded only to a center portion on an upper surface of an outer peripheral wall portion that defines the recess portion of the substrate, and
a material of the metal thin film is any one of Pd, a Pd alloy, a Pd—Cu alloy, and TiN.
